(19) United States
(12) Patent Application Publication
(10) Pub. No.: US 2009/0163544 A1
(43) Pub. Date: Jun. 25, 2009

(54) Use of NK-3 Receptor Antagonists for the Treatment of Nausea and Vomiting

(76) Inventors: Michael Rigby, Saffron Walden (GB); Frederick David, Bishop Stortford (GB)

Correspondence Address: MERCK AND CO., INC, P O BOX 2000, RAHWAY, NJ, 07065-0907, US

(21) Appl. No.: 11/887,670

(22) PCT Filed: Mar. 31, 2006
(86) PCT No.: PCT/GB2006/050070

§ 371 Date: Oct. 31, 2008

(30) Foreign Application Priority Data
Apr. 4, 2005 (GB) 0506800.2

(51) Int. Cl. A61K 31/47; A61K 31/4545

(52) U.S. Cl. 514/312; 514/316

(57) ABSTRACT

This invention relates to the treatment or prevention of nausea and/or vomiting by the administration of a NK-3 receptor antagonist.

This invention relates to the treatment or prevention of nausea and/or vomiting by the administration of NK-3 receptor antagonists.

Nausea and vomiting may be drug-induced (e.g. chemotherapy, premedication, anaesthesia), surgical laparotomy (including ophthalmic and abdominal surgery), motion-induced (e.g. travel sickness, vertigo, labyrinthitis), a normal physiological response (e.g. following ingestion of contaminated food) or an abnormal physiological response (e.g. in pregnancy).

Certain drug treatments, such as cancer chemotherapy, antibiotics, treatment for erectile dysfunction (e.g. apomorphine) and analgesics such as opioids (e.g. morphine, alfentanil) can cause nausea and vomiting. Nausea is particularly difficult to control and may be more debilitating then vomiting. Indeed existing treatments only provide poor control of nausea when compared with the control of vomiting.

Drugs useful in the treatment of nausea and/or vomiting include antagonists at NK-1 (e.g. aprepitant), 5-HT3 (e.g. ondansetron, tropisetron, granisetron, palosetron, dolasetron), dopamine (e.g. domperidone, metoclopramide, droperidol, prochlorperizine, perphenazine, promethazine, trifluoperazine, chlorpromazine), histamine (promethazine, dimenhydrinate, cyclizine, cinnarizine, betahistine) and muscarinic (e.g. hyoscine, also known as scopolamine) and cannabinoid (Delta tetrahydrocannabinol, nabilone) receptors. However, side effects are common with these classes of agents, including sedation (dopamine, histamine and muscarinic antagonists), extrapyramidal side effects (dopamine antagonists), urinary retention and blurred vision (muscarinic antagonists) and constipation (5-HT3 antagonists).

Neurokinin 3 (NK-3) receptor antagonists are being developed for the treatment of a number of physiological disorders mediated by tachykinins. Examples of conditions in which the NK-3 receptor has been implicated include disorders of the central nervous system, such as schizophrenia, and in the periphery, such as irritable bowel disease, pre-eclampsia, Chronic Obstructive Pulmonary Disease (COPD) and asthma.

Background information on NK-3 receptor antagonists can be found in literature reviews such as Giardina and Raveglia, Exp. Opin. Ther. Patents (1997) 7(4): 307-323 and Giardina et al., Exp. Opin. Ther. Patents (2000) 10(6): 939-960. These references also contain pertinent information on preclinical validation of therapies that can be treated with NK-3 antagonists.

Representative examples of compounds prepared in the art as NK-3 antagonists are to be found in WO-A-9719926 (SmithKline Beecham S.p.a) and U.S. Pat. No. 5,741,910 (Sanofi). Structurally related compounds as NK-3 and/or NK-2 receptor antagonists are disclosed in published International patent application WO 2004/072045.

Nevertheless, there is a need for novel treatments with fewer side effects and increased efficacy for nausea and/or vomiting.

The present invention provides the use of a NK-3 receptor antagonist, or a pharmaceutically acceptable salt thereof, for the treatment of nausea and/or vomiting. The compounds of this class advantageously provide better control and the ability to lower the dose of existing anti-emetic/anti-nausea agents when given as combination therapy when compared against alternative nausea and vomiting therapies.

The present invention also provides the use of a NK-3 receptor antagonist, or a pharmaceutically acceptable salt thereof, for the manufacture of a medicament for the treatment of nausea and/or vomiting.

The present invention further provides a method for the treatment of nausea and/or vomiting, which method comprises administration to a patient in need of such treatment of an effective amount of a NK-3 receptor antagonist, or a pharmaceutically acceptable salt thereof.

In a further aspect of the present invention, there is provided a pharmaceutical composition for the treatment of nausea and/or vomiting which comprises a NK-3 receptor antagonist, or a pharmaceutically acceptable salt thereof, together with a pharmaceutically acceptable carrier or excipient.

The NK-3 receptor antagonist may be used alone or in combination with one or more other NK-3 receptor antagonists.

Whilst it is envisaged that a NK-3 receptor antagonist will be useful alone in the treatment of nausea and/or vomiting, it will be appreciated that a combination of a NK-3 receptor antagonist with one or more other anti-emetic and/or anti-nauseant agents may provide an enhanced effect in the treatment of nausea and/or vomiting. Such a combination would be expected to provide more complete control. Furthermore, such a combination may enable a lower dose of the active agents to be used, thereby minimising the risk of adverse side-effects.

Thus, according to a further aspect of the present invention, there is provided a combination comprising a NK-3 receptor antagonist, or a pharmaceutically acceptable salt thereof, with an anti-emetic and/or an anti-nauseant agent.

There is also provided the use of a NK-3 receptor antagonist, or a pharmaceutically acceptable salt thereof, and an anti-emetic and/or an anti-nauseant drug for the manufacture of a medicament for the treatment of nausea and/or vomiting.

The present invention also provides a method for the treatment of nausea and/or vomiting, which method comprises administration to a patient in need of such treatment of an amount of a NK-3 receptor antagonist, or a pharmaceutically acceptable salt thereof, and an amount of an anti-emetic and/or an anti-nauseant agent, such that together they give effective relief.

In a further aspect of the present invention, there is provided a pharmaceutical composition comprising a NK-3 receptor antagonist, or a pharmaceutically acceptable salt thereof, and an anti-emetic and/or an anti-nauseant agent, together with at least one pharmaceutically acceptable carrier or excipient.

It will be appreciated that an agent displaying dual anti-emetic and anti-nausea effects, such as NK-1 receptor antagonists will also be suitable for use where an anti-emetic and/or an anti-nauseant agent is required.

It will also be appreciated that certain NK-3 receptor antagonists will have antagonist activity at the NK-2 receptor or the NK-1 receptor or both the NK-2 and NK-1 receptors, and these are also suitable for use in the present invention.

It will also be appreciated that the NK-3 receptor antagonist and the anti-emetic and/or anti-nauseant agent may be present as a combined preparation for simultaneous, separate or sequential use for the treatment of nausea and/or vomiting. Such combined preparations may be, for example, in the form of a twin pack.

In a further or alternative aspect of the present invention, there is therefore provided a product comprising a NK-3 receptor antagonist, or a pharmaceutically acceptable salt thereof, and an anti-emetic and/or an anti-nauseant agent as a combined preparation for simultaneous, separate or sequential use in the treatment of nausea and/or vomiting.

It will be appreciated that when using a combination of the present invention, the NK-3 receptor antagonist and the anti-emetic and/or anti-nauseant agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially.

As used herein, the term “treatment” refers both to the treatment and to the prevention or prophylactic therapy of the aforementioned conditions.

NK-3 receptor antagonists may be identified using the tests disclosed in published US patent application no. US2004/002504 Merck Sharp & Dohme Limited).

Suitable NK-3 receptor antagonists for use according to the invention include, for example, those disclosed in published US patent application no. U.S. Pat. No. 5,741,910 (Sanofi) and published International patent application WO 97/19926 (SmithKline Beecham S.p.a).

Other suitable NK3 receptor antagonists include those disclosed in published International patent applications WO 2004/050626 and WO 2004/050627 (both SmithKline Beecham Corporation), WO 2004/072045 (Merck Sharp & Dohme Limited) and published US patent application no. US 2004/0002504 (Merck Sharp & Dohme Limited).

Particularly preferred NK-3 receptor antagonists of use in the present invention include those that are orally bioavailable and brain penetrant and have high affinity and long duration of action, such as osanetant, talnetant, triple (NK1/NK2/NK3) inhibitor SCH-206272, or a pharmaceutically acceptable salt thereof.

Full descriptions of the preparation of the NK-3 receptor antagonists which may be employed in the present invention may be found in the references cited herein.

Suitable anti-emetic and/or anti-nausea drugs of use in combination with a NK-3 receptor antagonist include NK-1 receptor antagonists, 5-HT3 receptor antagonists, muscarinic antagonists, anti-histamine drugs, dopamine antagonists, cannabinoids, anti-migraine drugs and anti-inflammatory drugs.

Examples of NK-1 receptor antagonists include those disclosed in published International patent applications WO 95/16679, WO 00/56727, WO 02/16344 and WO 03/022839, such as CP-99,994, CP-122721, CJ11974, GR205171, GR203040, PD154075 and R116301. A preferred NK-1 receptor antagonist of use in the present invention is 2-(R)-(1-S-3,5-bis(trifluoromethyl)phenyl)-2-hydroxyethoxy)-3-S)-(4-fluorophenyl)-4-(1,2,4-triazol-3-yl)methylmorpholine, or a pharmaceutically acceptable salt thereof.

Examples of 5-HT3 receptor antagonists include those disclosed by Gan (CNS Drugs 2005 19 (3) 225-238), Grunberg and Koeller (Exp Opin on Pharmacotherapy 2003, 4 (12), pp. 2297-2303), Jordan et al. (Eur J Cancer (2005) 41(2):199-205), Gregory and Ettinger (Drugs (1998);55(2):173-189) and Haus et al. (Scand J Rheumatol Suppl (2004) 119, 12-18), such as ondansetron, tropisetron, granisetron, palosetron and dolasetron.

An example of a muscarinic antagonist is hyoscine (scopolamine).

Examples of anti-histamines include promethazine, dimenhydrinate, cyclizine, cinnarizine and betahistine.

Examples of dopamine antagonists include amisulpride, aripiprazole, benperidol, chlorpromazine, clozapine, domperidone, droperidol, flupentixol, fluphenazine, haloperidol, levopromazine, metoclopramide, olanzapine, pericyazine, perphenazine, pimozide, prochlorperazine, promazine, promethazine, quietapine, remoxipride, risperidone, sertindole, sulpiride, trifluoroperazine, thioridazine, thiothixene, ziprasidone and zotepine.

Examples of cannabinoids include Δ-9-tetrahydrocannabinol (dronabinol), Δ-9,11-tetrahydrocannabinol, cannabinol and nabilone.

Examples of anti-migraine drugs include 5-HT1D antagonists, NSAIDs and ergotamine derivatives.

Examples of 5-HT1D antagonists include sumatriptan, rizatriptan, eletriptan, naratriptan, frovatriptan and zolmitriptan. Examples of NSAIDs include naproxen and ibuprofen.

Examples of anti-inflammatory drugs include steroids such as dexamethasone and prednisolone.

Suitable pharmaceutically acceptable salts of the NK-3 receptor antagonists of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid, p-toluenesulfonic acid or sulfuric acid. Salts of amine groups may also comprise the quaternary ammonium salts in which the amino nitrogen atom carries an alkyl, alkenyl, allynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

Suitable pharmaceutically acceptable salts of the anti-emetic or anti-nausea agents used in combination with a NK-3 receptor antagonist according to the present invention include those salts described above in relation to the salts of NK-3 receptor antagonists.

The compositions of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g. intravenous), intradermal, intramuscular, or intrathecal injection or infusion techniques and the like.

The pharmaceutical compositions can be in a form suitable for oral use, for example, as tablets, troches, lozenges, aqueous or oily suspensions, dispersible powders or granules, emulsions, hard or soft capsules, or syrups or elixirs. Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient or ingredients in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutically acceptable compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The compositions of the invention can also be administered in the form of suppositories, e.g., for rectal administration of the drugs. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Preferably, the compositions of the present invention are suitable for oral administration. Compositions in the form of tablets, pills, capsules or wafers for oral administration are particularly preferred.

The present invention further provides a process for the preparation of a pharmaceutical composition comprising a NK-3 receptor antagonist, or a pharmaceutically acceptable salt thereof, and an anti-emetic and/or anti-nausea agent, which process comprises bringing a NK-3 receptor antagonist, or a pharmaceutically acceptable salt thereof, and an anti-emetic and/or anti-nausea agent, into association with a pharmaceutically acceptable carrier or excipient.

When administered in combination, either as a single or as separate pharmaceutical composition(s), the NK-3 receptor antagonist and an anti-emetic and/or anti-nausea agent are presented in a ratio which is consistent with the manifestation of the desired effect. In particular, the ratio by weight of the NK-3 receptor antagonist and the anti-emetic and/or anti-nausea agent will suitably be between 0.001 to 1 and 1000 to 1, and especially between 0.01 to 1 and 100 to 1.

A minimum dosage level for the NK-3 receptor antagonist is about 1 mg per day, preferably about 5 mg per day and especially about 10 mg per day. A maximum dosage level for the NK-3 receptor antagonist is about 1500 mg per day, preferably about 1000 mg per day and especially about 500 mg per day. The compounds are administered one to three times daily, preferably once a day.

A minimum dosage level for the anti-emetic or anti-nausea agent will vary depending upon the choice of agent, but is typically about 0.5 mg per day for the most potent compounds or about 20 mg per day for less potent compounds. A maximum dosage level for the anti-emetic or anti-nausea agent is typically 30 mg per day for the most potent compounds or 200 mg per day for less potent compounds. The compounds are administered one to three times daily, preferably once a day.

It will be appreciated that the amount of the NK-3 receptor antagonist required for use in the treatment of nausea and/or vomiting will vary not only with the particular compounds or compositions selected but also with the route of administration, the nature of the condition being treated, and the age and condition of the patient, and will ultimately be at the discretion of the patient's physician or pharmacist.

When used in combination, it will be appreciated that the amount of the NK-3 receptor antagonist and the anti-emetic and/or anti-nausea agent required for use in the treatment of nausea and/or vomiting will vary not only with the particular compounds or compositions selected but also with the route of administration, the nature of the condition being treated, and the age and condition of the patient, and will ultimately be at the discretion of the patient's physician or pharmacist.

The following example illustrates a pharmaceutical composition according to the invention.

### EXAMPLE 1

### Tablets Containing 50-300 mg of NK-3 Antagonist

| | Amount mg | | |
|---|---|---|---|
| NK-3 antagonist | 50.0 | 100.0 | 300.0 |
| Microcrystalline cellulose | 80.0 | 80.0 | 80.0 |
| Modified food corn starch | 80.0 | 80.0 | 80.0 |
| Lactose | 189.5 | 139.5 | 139.5 |
| Magnesium Stearate | 0.5 | 0.5 | 0.5 |

The active ingredient, cellulose, lactose and a portion of the corn starch are mixed and granulated with 10% corn starch paste. The resulting granulation is sieved, dried and blended with the remainder of the corn starch and the magnesium stearate. The resulting granulation is then compressed into tablets containing 50 mg, 100 mg and 300 mg of the NK-3 receptor antagonist per tablet.

1-12. (canceled)

13. A method for the treatment of nausea or vomiting, which method comprises administration to a patient in need of such treatment of an effective amount of a NK-3 receptor antagonist, or a pharmaceutically acceptable salt thereof.

14. The method of claim 13 which additionally comprises administration of an anti-emetic or an anti-nauseant agent.

15. A method of claim 13 for the treatment of nausea or vomiting, which method comprises administration to a patient in need of such treatment of an amount of a NK-3 receptor antagonist, or a pharmaceutically acceptable salt thereof, and an amount of an anti-emetic or an anti-nauseant agent, such that together they give effective relief.

16. The method of claim 13 wherein the NK-3 receptor antagonist is selected from osanetant, talnetant, SCH-206272, or a pharmaceutically acceptable salt thereof.

17. The method of claim 14 wherein the anti-emetic or anti-nausea agent is selected from an NK-1 receptor antagonist, 5-HT3 receptor antagonist, muscarinic antagonist, anti-histamine drug, dopamine antagonist, cannabinoid, anti-migraine drug and anti-inflammatory drug.
